SEQUENCE LISTING (7 sequences)

SEQ ID NO: 1 - DNA, 20 bp, Artificial Sequence
  synthetic cytokeratin 5 (CK5), keratin 5, keratin, type II cytoskeletal 5 (KRT5, K5, KRT5A), epidermolysis bullosa simplex 2 (EBS2), Dowling-Meara/Kobner/Weber-Cockayne types (DDD) PCR intron-spanning primer hCK5spanf
cttgtggagtgggtggctat

SEQ ID NO: 2 - DNA, 20 bp, Artificial Sequence
  synthetic cytokeratin 5 (CK5), keratin 5, keratin, type II cytoskeletal 5 (KRT5, K5, KRT5A), epidermolysis bullosa simplex 2 (EBS2), Dowling-Meara/Kobner/Weber-Cockayne types (DDD) PCR intron-spanning primer hCK5spanf
ccacttggtgtccagaacct

SEQ ID NO: 3 - DNA, 20 bp, Artificial Sequence
  synthetic housekeeping gene glyceraldehyde phosphate dehydrogenase (GAPDH) internal loading control PCR intron-spanning primer GAPDHf
ggagtcaacgggtatttggt

SEQ ID NO: 4 - DNA, 20 bp, Artificial Sequence
  synthetic housekeeping gene glyceraldehyde phosphate dehydrogenase (GAPDH) internal loading control PCR intron-spanning primer GAPDHr
gacaagcttcccgttctcag

SEQ ID NO: 5 - DNA, 19 bp, Artificial Sequence
  synthetic Taqman forward primer positioned over exon-exon junction
ttctttgatgcggagctgt

SEQ ID NO: 6 - DNA, 20 bp, Artificial Sequence
  synthetic Taqman reverse primer positioned over exon-exon junction
catggagaggaccactgagg

SEQ ID NO: 7 - DNA, 24 bp, Artificial Sequence
  synthetic Taqman probe
cccagatgcagacgcatgtctctg

(19) United States
(12) Patent Application Publication
(10) Pub. No.: US 2009/0291445 A1
(43) Pub. Date: Nov. 26, 2009

(54) BIOMARKER OF LUNG INJURY AND REPAIR

(75) Inventors: Brigitte N. Gomperts, Los Angeles, CA (US); Derek W. Nickerson, West Hollywood, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 12/423,329

(22) Filed: Apr. 14, 2009

Related U.S. Application Data
(60) Provisional application No. 61/044,726, filed on Apr. 14, 2008.

(51) Int. Cl. C12Q 1/68

(52) U.S. Cl. 435/6

(57) ABSTRACT

The present invention resides in the discovery that circulating cytokaretin 5 (CK5) mRNA level correlates with the presence of a lung injury or disease as well as the severity or stage of the injury or disease. Diagnostic methods and kits are provided.

### CROSS REFERENCE TO RELATED APPLICATIONS

This application claims priority to U.S. Provisional Application No. 61,044,726, filed Apr. 14, 2008, the contents of which are incorporated herein by reference in the entirety.

### STATEMENT OF GOVERNMENT RIGHTS

This application is based on research supported in part by the National Institutes of Health Grant No. HL074229, the U.S. Government therefore has certain rights to this invention.

### BACKGROUND OF THE INVENTION

Circulating epithelial progenitor cells are important for repair of the airway epithelium in a mouse model of tracheal transplantation. The present inventors investigated whether circulating epithelial progenitor cells would also be present in normal human subjects and could be important for repair of the airway after lung injury. As lung transplantation is associated with lung injury, which is severe early on and exacerbated during episodes of infection and rejection, the inventors particularly investigated whether circulating epithelial progenitor cell levels could predict clinical outcome following lung transplantation. By Quantitative Real Time Polymerase Chain Reaction (PCR), peripheral blood mRNA levels were determined for cytokeratin 5 (CK5), a previously characterized marker of circulating epithelial progenitor cells, in healthy human subjects, in lung transplant recipients immediately post-transplant and serially thereafter, and in heart transplant recipients. All normal human subjects examined were found to express cytokeratin 5 in their buffy coat in amounts that were not significantly influenced by age or gender. A profound, statistically significant decrease in cytokeratin 5 mRNA expression levels was found in lung transplant patients, when compared to healthy human subjects (p=3.1×10⁻¹³) and to heart transplant recipients. Lung transplant recipients with recovering lung function, as measured by improved FEV1 values, exhibited improved circulating cytokeratin 5 levels (p=0.03). Revealing that levels of cytokeratin 5 mRNA, a proxy marker for circulating epithelial progenitor cells, are inversely correlated with disease or injury status of the lungs in patients, especially lung transplant recipients, this invention therefore provides a new method for detecting lung disorders in patients, as well as for monitoring lung functions and predicting clinical outcome in patients with lung injuries or in post-transplant patients.

On the other hand, lung cancer is the most common cancer worldwide and a highly lethal disease, with most patients being diagnosed at late, inoperable stage disease and having a very poor prognosis. By far the leading cause of cancer death among both men and women, lung cancer accounts for more deaths than prostate cancer, breast cancer, and colon cancer combined. The current 5-year survival rate for all stages of lung cancer is only 15%. Since early stage diagnosis provides a better prognosis, new methods are desired for the early detection of this deadly disease. The present inventors discovered that the level of circulating CK5 mRNA in a patient is correlated with the presence of lung cancer or the progressive stage of the disease: lung cancer patients have lower levels of CK5 mRNA when compared with healthy individuals without lung cancers, and patients with late stage lung cancer have particularly low levels of circulating CK5 mRNA. CK5 therefore can be used as a biomarker for indicating the presence of lung cancer in a patient, or for indicating whether the lung cancer in a patient has progressed to a late stage or remains in an early stage. This discovery thus fulfills the need for early detection/monitoring of lung cancer and other related needs.

### BRIEF SUMMARY OF THE INVENTION

In one aspect, this invention provides a method for detecting a lung disorder in a patient. The method comprises the steps of: (i) quantitatively determining the amount of cytokeratin 5 (CK5) mRNA in the patient's blood; and (ii) comparing the amount of CK5 mRNA from step (i) to a standard control representing the amount of CK5 mRNA in the blood of an average healthy person without any lung disorder, wherein an increase or decrease in the amount of CK5 mRNA from the standard control indicates the presence of a lung disorder. Typically, the CK mRNA is extracted from the buffy coat of a patient's blood sample. In some cases, step (i) is performed by reverse transcriptase polymerase chain reaction (RT-PCR).

In some embodiments, a decrease in the amount of CK5 mRNA from the standard control is observed in step (ii) and indicates a lung disorder such as an injury of the lung, an infection or inflammation of the lung, or lung cancer, or a late stage lung cancer. In some cases, the decrease in the amount of CK5 mRNA from the standard control is more than 20%, 50%, 75%, or more than 99% or even higher. In some cases, the patient being tested has just received a lung transplant.

In a second aspect, the present invention provides a kit for diagnosing or monitoring a lung disorder in a patient. The kit includes these components: (i) PCR primers for quantitatively determining the amount of CK5 mRNA in the patient's blood; and (ii) a standard control representing the amount of CK5 mRNA an average healthy person without any lung disorder. Optionally, the kit also contains a user's instructions on how to use the kit, such as for detecting and/or monitoring a lung disorder including an injury of the lung, a lung transplant, or a lung cancer.

### BRIEF DESCRIPTION OF THE DRAWINGS

FIG. 1A. PCR for CK5 mRNA from the circulation of healthy volunteers and lung transplant patients. The top panel shows the expected 439 bp fragment for CK5 using cDNA as template in healthy volunteers (Lanes 1-4) and CK5 mRNA expression from a representative group of patients post lung transplantation (Lanes 5-9). CK5 mRNA expression was not found in PCR Lanes 5, 8, and 9 and neither was CK5 mRNA expression detectible by quantitative real-time PCR in these samples. Lane 10 represents the positive control, which consists of a bacterial artificial chromosome (BAC) containing 170 kb of genomic sequence surrounding the CK5 locus as template. Lane 11 is the negative control without cDNA template. The bottom panel shows PCR amplification of GAPDH from the same templates. FIG. 1B. Standard curve of real-time PCR amplification of CK5 and GAPDH. The log quantity of RNA is plotted against the mean threshold cycle (Ct), measured in triplicate. Slope and intercept are represented in the equations of the regression lines, along with the regression coefficient.

FIG. 2A. Quantitative real-time PCR of CK5 plotted against age of normal human subjects. A scatter plot of data shows no significant difference between the log of the age and CK5 mRNA expression levels in all 38 normal human subjects examined (p=0.273). FIG. 2B. Quantitative real-time PCR expression of CK5 does not differ with gender. A box plot demonstrates the similarity in CK5 levels in male and female normal human subjects (p=0.84).

FIG. 3. Quantitative real-time PCR of CK5 expression in normal human subjects compared to lung transplant patients and heart transplant patients. A box plot demonstrates the differences between CK5 values in normal human subjects compared to lung transplant patients (p=3.1×10⁻¹³). A further comparison is made between CK5 mRNA expression in the circulation of heart transplant patients (n=6) and lung transplant patients (p=0.004).

FIG. 4A. Quantitative real-time PCR of CK5 expression in lung transplant patients correlated with time post-transplant. This scatter plot shows the increase in CK5 mRNA expression (decrease in ΔCt for CK5) with time post transplant. The X-axis represents set time points post transplant (0=time of transplant; 1=1 day post-transplant (PT); 2=1 week PT; 3=1 month PT; 4=3 months PT; 5=6 months PT). FIG. 4B. Percentage decrease in FEV1 in lung transplant patients correlated with time post-transplant. Scatter plot of FEV1 on the Y-axis with 0.0 being 100% FEV1 compared to time post transplant on the X-axis (0=time of transplant; 1=1 day PT; 2=1 week PT; 3=1 month PT; 4=3 months PT; 5=6 months PT). FIG. 4C. Quantitative real-time PCR of CK5 expression in lung transplant patients correlated with percentage decrease in FEV1 post-transplant.

FIG. 5. Quantitative Real-Time PCR for CK5 from blood samples from normal human subjects and patients with lung cancer. We found a significant difference between levels of CK5 expression in the circulation of normal human subjects and lung cancer patients, with lung cancer patients having lower expression of CK5 in their circulation (p<0.0001). Note the y-axis represents ACT for CK5 and normalizes for GAPDH as a loading control. Therefore, the lower the value for ΔCT for CK5, the higher the CK5 mRNA expression in the circulation.

FIG. 6. Quantitative Real-Time PCR for CK5 from blood samples from normal human subjects and patients with lung cancer. Comparison of early (stage I/II) versus late (stage III/IV) stage lung cancer patients demonstrated lower expression of CK5 mRNA in the circulation of late stage lung cancer patients compared to early stage patients (p=0.03). There is therefore a gradation of levels of CEPC, with healthy normal human subjects having the most CEPC, early stage lung cancer patients have less expression and late stage lung cancer patients have the least expression (p=0.005).

### DEFINITIONS

As used herein, the term “cytokeratin 5” or “CK5” refer to nucleic acids, e.g., gene, pre-mRNA, mRNA, and polypeptides, polymorphic variants, alleles, mutants, and interspecies homologs that: (1) have an amino acid sequence that has greater than about 60% amino acid sequence identity, or greater than about 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, preferably up to the entire length, of a polypeptide encoded by a respectively referenced nucleic acid or an amino acid sequence described herein, for example, as depicted in GenBank Accession Nos. NM_{—}000424 (human CK5 mRNA) and NP_{—}000415 (human CK5 protein); (2) specifically bind to antibodies, e.g., polyclonal antibodies, raised against an immunogen comprising a referenced amino acid sequence as depicted in GenBank Accession No. NP_{—}000415 (human CK5 protein); immunogenic fragments respectively thereof, and conservatively modified variants respectively thereof, (3) specifically hybridize under stringent hybridization conditions to a nucleic acid encoding a referenced amino acid sequence as depicted in GenBank Accession No. NP_{—}000415 (human CK5 protein), respectively, and conservatively modified variants respectively thereof, (4) have a nucleic acid sequence that has greater than about 95%, preferably greater than about 96%, 97%, 98%, 99%, or higher nucleotide sequence identity, preferably over a region of at least about 25, 50, 100, 150, 200, 250, 500, 1000, or more nucleotides, to a reference nucleic acid sequence as shown in GenBank Accession No. NM_{—}000424 (human CK5 mRNA). A polynucleotide or polypeptide sequence is typically from a mammal including, but not limited to, primates (e.g., human), rodents (rat, mouse, hamster, etc.), cows, pigs, horses, sheep/goats, or any other mammals. The CK5 nucleic acids and proteins useful for the invention include both naturally occurring or recombinant molecules.

The term “lung disorder” as used herein refers to any disease or injury to the lung tissue that causes the lung to function improperly, including but not limited to, infection or inflammation, physical trauma, or transplant of the lung tissue. Lung cancers of all types are also within the meaning of the term “lung disorders” Three main types of lung diseases encompassed by this term include obstructive lung disease—a decrease in the exhaled air flow caused by a narrowing or blockage of the airways, which can occur with asthma, emphysema, and chronic bronchitis; restrictive lung disease—a decrease in the total volume of air that the lungs are able to hold, often related to a decrease in the elasticity of the lungs themselves; and a defect in the ability of the lung's air sac tissue to move oxygen into a person's blood. Some examples of lung disorders are: asthma; chronic bronchitis; COPD (chronic obstructive pulmonary disease); emphysema; interstitial lung disease; pulmonary fibrosis; and sarcoidosis. Other lung disorders including lung injuries caused by environmental factors are: asbestosis; aspergilloma; aspergillosis; aspergillosis—acute invasive; atelectasis; eosinophilic pneumonia; lung cancer, especially metastatic lung cancer; necrotizing pneumonia; pleural effusion; pneumoconiosis; pneumocystosis; pneumonia, especially pneumonia in immunodeficient patients; pneumothorax; pulmonary actinomycosis; pulmonary alveolar proteinosis; pulmonary anthrax; pulmonary arteriovenous malformation; pulmonary edema; pulmonary embolus; pulmonary histiocytosis X (eosinophilic granuloma); pulmonary hypertension; pulmonary nocardiosis; pulmonary tuberculosis; pulmonary veno-occlusive disease; and rheumatoid lung disease, etc.

The term “average,” as used in the context of describing a healthy person, i.e., one who is not suffering from a lung disorder or at risk of developing a lung disorder, refers to certain characteristics, such as the level of CK5 mRNA found in the person's blood, that are representative of a randomly selected group of individuals not suffering from or at risk of developing any lung disorder. This selected group typically comprises a sufficient number of individuals such that the average level of CK5 mRNA among these individuals reflects, with reasonable accuracy, the level of CK5 mRNA in the general population of healthy individuals free of lung disorders. In addition, the selected group of individuals may, optionally, have similar aspects in medical history, such as in age, gender, ethnic background, etc.; while in other cases no such similarity is required for establishing an average amount of circulating CK5 mRNA.

“Standard control” as used herein refers to a sample suitable for the use as a comparison basis in a method of the present invention, in order for determining whether an increase or decrease exists in the amount of CK5 mRNA found in a patient's blood. Such sample contains a known amount of the CK5 mRNA that closely reflects the average level of CK5 mRNA in an average individual who is not suffering from a lung disorder or at risk of developing a lung disorder, as described above.

“An increase or a decrease in the amount of mRNA from the standard control” as used herein refers to a positive or negative change in amount from the standard control. An increase is typically at least 10%, or at least 20%, or 50%, or 2-fold, or at least 5-fold, and can be as high as at least 10-fold or even 20-fold. Similarly, a decrease is typically at least 50%, or at least 80%, or at least 90%, or even as high as more than 99% in reduction from the level of standard control.

A “polynucleotide hybridization method” as used herein refers to a method for detecting the presence and/or quantity of a polynucleotide based on its ability to form Watson-Crick base-pairing, under appropriate hybridization conditions, with a polynucleotide probe of a known sequence. Examples of such hybridization methods include Southern blotting and Northern blotting.

“PCR primers” as used herein refer to oligonucleotides, typically in pairs, that can be used in a polymerase chain reaction (PCR) to amplify a nucleotide sequence originated from an mRNA encoding a protein of interest, such as human CK5. Typically, at least one of the PCR primers for amplification of a nucleotide sequence encoding a CK5 protein should be sequence-specific for the protein.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., John Wiley & Sons.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

A “biological sample” includes any section of tissues such as biopsy and autopsy samples, and frozen sections taken for histological purposes. Such samples include blood and blood fractions or products (e.g., serum, plasma, platelets, red blood cells, and the like), sputum, tissue, cultured cells, e.g., primary cultures, explants, and transformed cells, stool, urine, etc. A biological sample is typically obtained from a eukaryotic organism, most preferably a mammal such as a primate (e.g., chimpanzee or human); cow; dog; cat; rodent (e.g., guinea pig, rat, or mouse); rabbit; bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (i.e., prostate, lymph node, liver, bone marrow, blood cell), the size and type of the tumor (i.e., solid or suspended (i.e., blood or ascites)), among other factors. Representative biopsy techniques include excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

### DETAILED DESCRIPTION OF THE INVENTION

### I. Introduction

The proximal airway epithelium is in contact with the environment and, as such, is at constant jeopardy from environmental injury. An efficient mechanism for airway repair is therefore essential to protect the host. The current understanding of proximal airway repair is that a progenitor cell pool is located in the submucosal glands and submucosal gland ducts that are capable of self renewal and of differentiating in to the proximal airway subtypes, e.g., mucus and ciliated cells (Engelhardt et al. (1995) Development 121: 2031-2046; Borthwick et al. (2001) Am J Respir Cell Mol Biol 24: 662-670; Hong et al. (2004) Am J Physiol Lung Cell Mol Physiol 286: L643-649; Hong et al. (2004) Am J Pathol 164: 577-588; Schoch et al. (2004) Am J Physiol Lung Cell Mol Physiol 286: L631-642). These progenitor cells express the immature cytokeratins (CK) CK5 and CK14 and move up the submucosal gland ducts to form the basal layer of the pseudostratified columnar epithelium of the proximal airway. From there the basal cells lose CK5/14 and gain more mature cytokeratins, e.g., CK8/18 as they differentiate and move apically.

The present inventors have previously demonstrated the presence of circulating CK5 expressing cells that contributed to airway repair in a mouse model of ischemic injury and proximal airway repair. FACS analysis was used to show the presence of CK5 expressing cells in the bone marrow and circulation of mice (Gomperts et al. (2006) J Immunol. 176: 1916-1927). The identification of circulating epithelial cells that contribute to airway repair represents a controversial paradigm shift in the current concept of airway repair and regeneration after injury. The inventors have now discovered that CK5 mRNA expression could be quantified in the circulation of normal human subjects and CK5 mRNA levels are altered with severe airway disease, such as in lung transplant patients with end stage lung disease. CK5 mRNA expression level therefore can function as a clinical biomarker of airway disease.

The present invention provides, for the first time, methods and kits for detecting or diagnosing a lung disorder in a patient, by analyzing the level of CK5 mRNA present in the patient's blood. The same general methodology is also useful for monitoring the progression or severity of the lung disorder based on the changes in level of CK5 mRNA circulating the person's blood. According to the invention, the amount of CK5 mRNA in a blood sample can be quantitatively determined, preferably following an amplification procedure, e.g., reverse transcriptase polymerase chain reaction (RT-PCR). The amount of CK5 mRNA is then compared to a standard control having a CK5 mRNA level that is representative of an average person without any lung disorders. Typically, a decrease from the standard level of CK5 mRNA indicates the presence of a lung disorder or an increased risk of developing a lung disorder, and the larger the decrease, the more severe the disorder or the later stage the disorder has progressed to. On the other hand, if, during the course of a lung disorder progression, the reduced level of CK5 mRNA is gradually rising up and becoming closer to the standard control level, i.e., the decrease or deficiency in the patient's CK5 mRNA level when compared with the standard control is diminishing, that indicates an improved lung function or lessening of the disease in the patient. In the cases of detecting and monitoring lung cancers, a decrease in the circulating CK5 mRNA level from the standard control indicates the presence of lung cancer, and a larger decrease often indicates that the lung cancer has developed to a later stage.

The present invention thus provides a novel approach for diagnosing a lung disorder, and for monitoring the progression of a lung disorder. As a medical professional will recognize, the diagnostic/monitoring method of this invention is often used in connection with other known diagnostic criteria for specific conditions under consideration, e.g., post-transplant lung injury, lung cancer, etc., for an accurate and meaningful assessment of the patient's condition.

### II. Preparation of Biological Samples

### A. Obtaining Biological Samples

The first step of practicing the present invention is to obtain a biological sample, e.g., a blood sample, from a patient for testing or monitoring lung function using a method of the present invention. The specific methods for taking biological samples vary depending on the site or sites where the samples are taken. Standard procedures routinely employed in hospitals or clinics are typically followed for this purpose. For example, collection of blood samples from a patient is performed on a daily basis in a medical office. An appropriate amount of sample, e.g., between 5 to 20 ml of peripheral blood, is collected and maybe stored according to standard medical laboratory testing procedure prior to further preparation.

### B. Preparing Blood Samples for RNA Extraction

The buffy cost of a person's blood is the suitable source of CK5 mRNA for testing in accordance with the present invention and can be obtained by well known methods. Buffy coat is the upper, lighter portion of the blood clot occurring when coagulation is delayed or when blood has been centrifuged. For example, a patient's blood can be placed in a tube containing EDTA or a specialized commercial product such as Vacutainer SST (Becton Dickinson, Franklin Lakes, N.J.) to prevent blood clotting, and, following centrifugation, buffy cost can be obtained as the thin layer located in between a layer of clear fluid (plasma) and a layer of red fluid containing most of the red blood cells. Centrifugation is typically conducted at an appropriate speed, e.g., 1,500-3,000×g, in a chilled environment, e.g., at a temperature of about 4-10° C.

### III. Quantitative Determination of CK5 mRNA

A. Extraction of mRNA

There are numerous methods for extracting mRNA from a biological sample, e.g., buffy coat of a blood sample. The general methods of mRNA preparation (e.g., described by Sambrook and Russell, Molecular Cloning: A Laboratory Manual 3d ed., 2001) can be followed; various commercially available reagents or kits, such as Trizol reagent (Invitrogen, Carlsbad, Calif.), Oligotex Direct mRNA Kits (Qiagen, Valencia, Calif.), RNeasy Mini Kits (Qiagen, Hilden, Germany), and PolyATtract® Series 9600™ (Promega, Madison, Wis.), may also be used to obtain mRNA from a blood sample from a woman. Combinations of more than one of these methods may also be used.

It is essential that all contaminating DNA be eliminated from the RNA preparations. Thus, careful handling of the samples, thorough treatment with DNase, and proper negative controls in the amplification and quantification steps should be used.

B. PCR-Based Quantitative Determination of mRNA Level

Once mRNA is extracted from a biological sample, e.g., a blood sample, the amount of CK5 mRNA may be quantified. The preferred method for determining the mRNA level is an amplification-based method, e.g., by PCR.

Prior to the amplification step, a DNA copy (cDNA) of the mRNA of interest must be synthesized. This is achieved by reverse transcription, which can be carried out as a separate step, or in a homogeneous reverse transcription-polymerase chain reaction (RT-PCR), a modification of the polymerase chain reaction for amplifying RNA. Methods suitable for PCR amplification of ribonucleic acids are described by Romero and Rotbart in Diagnostic Molecular Biology: Principles and Applications pp. 401-406; Persing et al., eds., Mayo Foundation, Rochester, Minn., 1993; Egger et al., J. Clin. Microbiol. 33:1442-1447, 1995; and U.S. Pat. No. 5,075,212.

The general methods of PCR are well known in the art and are thus not described in detail herein. For a review of PCR methods, protocols, and principles in designing primers, see, e.g., Innis, et al., PCR Protocols: A Guide to Methods and Applications, Academic Press, Inc. N.Y., 1990. PCR reagents and protocols are also available from commercial vendors, such as Roche Molecular Systems.

PCR is most usually carried out as an automated process with a thermostable enzyme. In this process, the temperature of the reaction mixture is cycled through a denaturing region, a primer annealing region, and an extension reaction region automatically. Machines specifically adapted for this purpose are commercially available.

Although PCR amplification of the target mRNA is typically used in practicing the present invention, one of skill in the art will recognize that amplification of the mRNA in a sample may be accomplished by any known method, such as ligase chain reaction (LCR), transcription-mediated amplification, and self-sustained sequence replication or nucleic acid sequence-based amplification (NASBA), each of which provides sufficient amplification. More recently developed branched-DNA technology may also be used to quantitatively determining the amount of mRNA markers in a biological sample. For a review of branched-DNA signal amplification for direct quantitation of nucleic acid sequences in clinical samples, see Nolte, Adv. Clin. Chem. 33:201-235, 1998.

### C. Other Quantitative Methods

The mRNA of interest can also be detected using other standard techniques, well known to those of skill in the art. Although the detection step is typically preceded by an amplification step, amplification is not required in the methods of the invention. For instance, the mRNA may be identified by size fractionation (e.g., gel electrophoresis), whether or not proceeded by an amplification step. After running a sample in an agarose or polyacrylamide gel and labeling with ethidium bromide according to well known techniques (see, Sambrook and Russell, supra), the presence of a band of the same size as the standard control is an indication of the presence of a target mRNA, the amount of which may then be compared to the control based on the intensity of the band. Alternatively, oligonucleotide probes specific to CK5 mRNA can be used to detect the presence of such mRNA species and indicate the amount of mRNA in comparison to the standard control, based on the intensity of signal imparted by the probe.

Sequence-specific probe hybridization is a well known method of detecting a particular nucleic acid comprising other species of nucleic acids. Under sufficiently stringent hybridization conditions, the probes hybridize specifically only to substantially complementary sequences. The stringency of the hybridization conditions can be relaxed to tolerate varying amounts of sequence mismatch.

A number of hybridization formats well known in the art, including but not limited to, solution phase, solid phase, or mixed phase hybridization assays. The following articles provide an overview of the various hybridization assay formats: Singer et al., Biotechniques 4:230, 1986; Haase et al., Methods in Virology, pp. 189-226, 1984; Wilkinson, In situ Hybridization, Wilkinson ed., IRL Press, Oxford University Press, Oxford; and Hames and Higgins eds., Nucleic Acid Hybridization: A Practical Approach, IRL Press, 1987.

The hybridization complexes are detected according to well known techniques and the detection is not a critical aspect of the present invention. Nucleic acid probes capable of specifically hybridizing to a target nucleic acid, i.e., the mRNA or the amplified DNA, can be labeled with a detectable label by any one of several methods typically used to detect the presence of hybridized nucleic acids. One common method of detection is the use of autoradiography using probes labeled with ³H, ¹²⁵I, ³⁵S, ¹⁴C, or ³²P, or the like. The choice of radioactive isotope depends on research preferences due to ease of synthesis, stability, and half lives of the selected isotopes. Other detection labels include compounds (e.g., biotin and digoxigenin), which bind to antiligands or antibodies labeled with fluorophores, chemilumi-nescent agents, and enzymes. Alternatively, probes can be conjugated directly with labels such as fluorophores, chemiluminescent agents or enzymes. The choice of label depends on sensitivity required, ease of conjugation with the probe, stability requirements, and available instrumentation.

The probes and primers necessary for practicing the present invention can be synthesized and labeled using well known techniques. Oligonucleotides used as probes and primers may be chemically synthesized according to the solid phase phosphoramidite triester method first described by Beaucage and Caruthers, Tetrahedron Letts., 22:1859-1862, 1981, using an automated synthesizer, as described in Needham-VanDevanter et al., Nucleic Acids Res. 12:6159-6168, 1984. Purification of oligonucleotides is by either native acrylamide gel electrophoresis or by anion-exchange HPLC as described in Pearson and Regnier, J. Chrom., 255:137-149, 1983.

### IV. Establishing a Standard Control

In order to establish a standard control, a group of individuals without any lung disorders is first be selected. These individuals may optionally have the same gender and similar age, ethnic background, and/or medical history. The lung function status of the selected individuals should be confirmed by well established, routinely employed methods, including but not limited to, X-ray, CT-scan, blood testing for possible infection/inflammation, and review of medical history.

Furthermore, the selected group of individuals without lung disorders should be of a reasonable size, such that the average amount of CK5 mRNA calculated from the group can be reasonably regarded as representative of the normal or average amount CK5 mRNA among the general population of healthy humans. Preferably, the selected group comprises at least 10 subjects.

Once an average value is established for the amount of mRNA encoding a CK5 protein based on the individual values found in each individual of the selected group, this values is considered a standard for the CK5 mRNA level for this type of sample. Any biological sample, e.g., a blood sample, that contains a similar amount of CK5 mRNA can thus be used as a standard control. A solution containing CK5 mRNA at a concentration of the established average of CK5 mRNA can also be artificially assembled and serve as a standard control.

### EXAMPLES

The following examples are provided by way of illustration only and not by way of limitation. Those of skill in the art will readily recognize a variety of non-critical parameters that could be changed or modified to yield essentially the same or similar results.

### Example 1

### CK5 mRNA in Lung Transplant Patients

### Methods

### Selection and Description of Participants

Ethics statement: The research was approved by the UCLA Institutional Review Board (IRB). IRB approval and informed written consent were obtained from all patients and normal human subjects examined. Data were analyzed anonymously.

Patients that were part of this study were patients at the University of California Los Angeles, and received lung transplants between 2005 and 2006. The patients in the study were placed on standard pre- and post-lung transplant immunosuppression and antimicrobials, as per standard of care. Only patients ≧18 years of age were included, as there is no pediatric lung transplant program at UCLA, otherwise all lung transplant recipients at UCLA were included.

Exclusion criteria included individuals involved in a clinical research trial utilizing an investigational therapy, pregnant women, lactating women, or women of childbearing age not willing to take precautions to avoid becoming pregnant during the study.

Subjects underwent peripheral blood collection just prior to lung transplantation. At 24 hours post lung transplant all recipients had their peripheral blood collected and then underwent a bronchoscopy with transbronchial biopsy. In addition, both peripheral blood and bronchoscopy with transbronchial biopsy were performed at 1 week, 4 weeks, 3 months, and 6 months post-transplantation and also when clinically indicated to evaluate for infection and/or rejection.

### Blood Samples

Venous blood samples were obtained from 23 lung transplant patients (aged 31-79 years) and 28 healthy volunteers (aged 19-54 years). All samples were collected in EDTA containing tubes. In the volunteer group, two 5-ml samples were collected and the first was discarded. This step was performed to eliminate possible contamination with epithelial cells from the epidermis during venipuncture. In the heart transplant group, samples were collected from central venous catheters.

RNA Extraction and cDNA Synthesis

### Lung Transplant Patients

Total RNA was isolated from buffy coat. Initially, buffy coat was collected from fresh whole blood following a 10 minute spin at 450×g. One (1) ml TRIzol Reagent (Invitrogen, Carlsbad, Calif.) and 0.2 volume chloroform was added per 5×10⁶ cells and the lysates were centrifuged at 12,000×g. RNA was collected in the aqueous phase. The RNA was then precipitated in isopropanol and washed with 70% ethanol. The resulting pellet was resuspended in 100 μl nuclease free water and further purified using the RNeasy Mini Kit (Qiagen, Valencia, Calif.) with on-column DNA digestion using the RNase-free DNase kit (Qiagen) as per the manufacturer's instructions.

### Volunteers and Heart Transplant Patients

Total RNA was extracted from a 1.5 ml aliquot of fresh whole blood using the RNeasy Blood Mini Kit (Qiagen). Manufacturer's instructions were followed except that following lysis of red blood cells, leukocytes were given one additional wash with buffer EL (Qiagen) to ensure removal of erythrocytes and any potential RT-PCR inhibitors. Samples were all treated on-column with the RNase-free DNase kit (Qiagen).

RNA concentration was measured in a Qubit fluorometer (Invitrogen). 1 μg of isolated total RNA was reversed transcribed with oligo-dT primers using the Taqman Reverse Transcription Reagents kit (Applied Biosystems N808-0234) in a final volume of 100 μl following the manufacturer's instructions. Some volunteer whole blood was also processed as described above for the Lung Transplant Patients and no difference was found in CK5 expression in the circulation when blood was processed fresh or frozen in TRIzol.

### Conventional PCR

PCR for CK5 and GAPDH was performed on cDNA template using intron-spanning primers. All primers are listed 5′-3′. Primers used were hCK5spanf (CTTGTGGAGTGGGTGGCTAT), hCK5spanR (CCACTTGGTGTCCAGAACCT) (CK5 GeneID: 3852), GAPDHf (GGAGTCAACGGGTATTTGGT) and GAPDHr (GACAAGCTTCCCGTTCTCAG). Cycling conditions were 95° C. for 5 min, 40 cycles of 95° C. for 30 sec, 56° C. for 30 sec, 72° C. for 30 sec and followed by a 5 min extension step at 72° C.

### Taqman Primer/Probe Design

Taqman primers and probe were designed using a web based tool from Genscript (website: genscript.com/ssl-bin/app/primer) with manual fine-tuning. The primer-probe set was selected so that the forward primer (TTCTTTGATGCGGAGCTGT) was positioned over an exon-exon junction. The reverse primer sequence is CATGGAGAGGACCACTGAGG. The resulting primer pair amplifies a 66 bp fragment. The Taqman probe is labeled at the 5′ end with a fluorescent dye (6-carboxy-fluorescein, FAM) and on the 3′ end with a quencher (6-carboxy-tetramethyl-rhodamine, TAMRA). Primers and probe were synthesized by IDT (Coralville, Iowa). The sequence of the probe is CCCAGATGCAGACGCATGTCTCTG. A Blast search (NCBI, NIH) showed no significant homology with other known genes in the database.

The housekeeping gene, glyceraldehyde phosphate dehydrogenase (GAPDH), was obtained as a pre-designed VIC labeled qPCR assay from Applied Biosystems (4310884E) (Foster City, Calif.) and used according to the manufacturer's instructions.

CK5 qPCR Assay Optimization and Validation

Optimal primer/probe concentrations were determined by first varying primer concentrations against the maximum recommended concentration of probe (250 nM). Subsequently, the concentration of probe was titrated. The concentration at which, for both primers and probe, the ΔCt and ΔRn did not change with increasing concentrations was selected.

Singleplex and multiplex qPCR was then performed to determine the compatibility of the CK5 assay with the assay for the internal control GAPDH. No competition between assays was observed, thus permitting the use of multiplex qPCR.

To determine the sensitivity and reproducibility of the assay, serial dilutions of cDNA template representing 5 log steps were prepared and run in triplicate.

Taqman qPCR Conditions

Real-time amplification and detection was performed with the ABI Step One Plus (Applied Biosystems) sequence detection system. Each reaction included 250 nM probe, 600 nM forward primer, 600 nM reverse primer, 7.5 μl Taqman Fast Universal PCR master mix (Applied Biosystems), nuclease free water, and, with the exception of the standard curve, 5 μl of cDNA corresponding to 50 ng of total RNA. Cycling conditions used were 20 sec at 95° C., and 40 cycles of 1 sec denaturation at 95° C. and 20 sec annealing at 60° C.

### Quantification and Statistics

The comparative Ct method for multiplex PCR was performed as outlined in the ABI PRISM 7700 Sequence Detection System User Bulletin #2 (available on the Applied Biosystems website: appliedbiosystems.com). Each sample was run in triplicate and amplified for the gene of interest CK5 and the housekeeping gene GAPDH. Samples that required more than 30 cycles to reach the threshold cycle (Ct) for GAPDH were discarded as it possibly indicated poor cDNA quality. Otherwise, the triplicate Ct values for each sample were averaged resulting in mean Ct values for both CK5 and GAPDH. The CK5 Ct values were then standardized to the housekeeping gene by taking the difference: ΔCt=Ct[CK5]−Ct[GAPDH]. In order to compare ΔCt values between cases and controls, the ΔCt values were averaged among the control samples and used the avg(ΔCt in controls) as a reference or calibrator ΔΔCt=ΔCt[sample]−avg(ΔCt controls). The fold-change between each sample and the reference sample was calculated as 2^{-ΔΔCt} for negative ΔΔCt values (which indicated an increase in fold change) and −2^{ΔΔCt} for positive ΔΔCt values (indicating a fold change decrease) (Livak and Schmittgen (2001) Methods 25: 402-408; Bookout and Mangelsdorf (2003) Nucl Recept Signal 1: e012).

Statistical analyses were primarily performed on ΔCt distributions as they were approximately normally distributed (while the fold change distributions were highly skewed). A Welch two-sample t-test was used to compare ΔCt distributions between approximately Gaussian distributions, and an exact Wilcoxon rank sum test was used for non-parametric distributions. Kendall rank correlation was used to test for associations between ΔCt values and other variables such as age and time. An exact Wilcoxon signed rank test was used to test for change in ΔCt values within patients over time. Mixed effects logistic regression models were used to study relationships between infection, rejection, and ΔCt. Scatterplots and box plots were helpful for visualizing the data and linear regression tools such as Cook's distance identified outliers. Bonferroni correction for multiple testing was considered in interpreting statistical significance. All analyses were performed using R, a free statistical program available at the website cran.r-project.org.

### Results

### Detection of CK5 in the Circulation of Normal Human Subjects and Patients by Conventional PCR

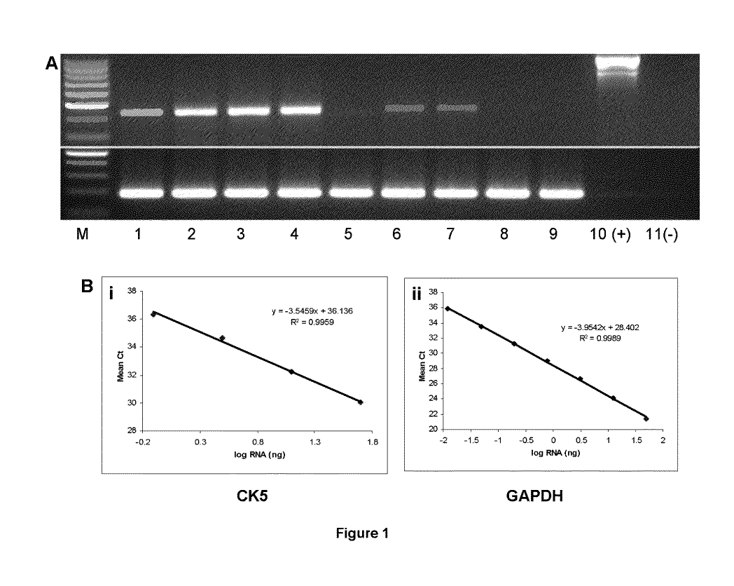

When conventional PCR was performed on cDNA obtained from the blood of normal human subjects and mRNA for CK5 was detected in all normal human subjects examined. PCR on lung transplant patient cDNA samples from the buffy coat revealed the presence of mRNA for CK5 in only some of the lung transplant patients. PCR with GAPDH primers was used to confirm the integrity of the cDNA (FIG. 1A).

### Validation of the Quantitative Real Time PCR Assay for CK5

The multiplex Quantitative Real Time PCR assay was first performed with varying concentrations of primer and then probe to determine the optimal concentration of primers and probe for the assay (Table 1). The concentration at which, for both primers and probe, the ΔCt and ΔRn did not change with increasing concentrations was selected. Thus primer concentrations of 600 nm and probe concentration of 250 nm were chosen. Then a standard curve consisting of serial dilutions of cDNA was performed with the determined CK5 primer and probe concentrations to determine the linear range of the assay. The triplicate reproducibility was noted to start to fall off the curve at threshold cycle (Ct) values greater than 36 (FIG. 1Bi). The linear range of the GAPDH assay is more extensive than the CK5 assay because of the abundance of GAPDH expression (FIG. 1Bii).

Quantification of the CK5 mRNA in Normal Human Subjects

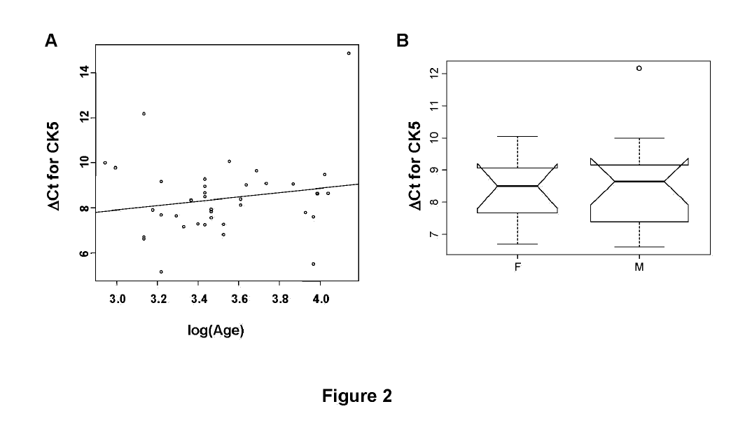

The triplicate Ct values for each sample were averaged resulting in mean Ct values for both CK5 and GAPDH. The CK5 Ct values were then standardized to the housekeeping gene by taking the difference: ΔCt=Ct[CK5]−Ct[GAPDH]. Most of the CK5 quantitative real-time PCR data were analyzed using the ΔCt variable, as it was approximately normally distributed in some sub groups of data. Kendall rank correlation was used to test for a ΔCt relationship with age in the control samples and an exact Wilcoxon rank sum test was used to compare ΔCt distributions between male and female control samples. Both tests yielded non-significant results (p-value=0.24 and 0.93, respectively). A scatterplot of ΔCt vs. log of age is shown in FIG. 2A. A boxplot of the sex-specific ΔCt values shows that there was no significant difference between males and females in our control data (FIG. 2B).

Longitudinal study of CK5 mRNA in Lung Transplant Patients

The inventors followed CK5 levels in a cohort of 23 lung transplant patients, ranging in age from 31-79 years that received transplants at the David Geffen School of Medicine at UCLA between February 2006 and March 2007. All patients had end stage lung disease at the time of transplant. About 30% of patients were transplanted for chronic obstructive pulmonary disease (COPD), about 30% for idiopathic pulmonary fibrosis (IPF) and the remaining for a variety of diagnoses. Most patients had 2-4 appointments within the year following their transplant where blood samples were drawn and their pulmonary function was tested. Table 2 details the lung transplant patient demographics.

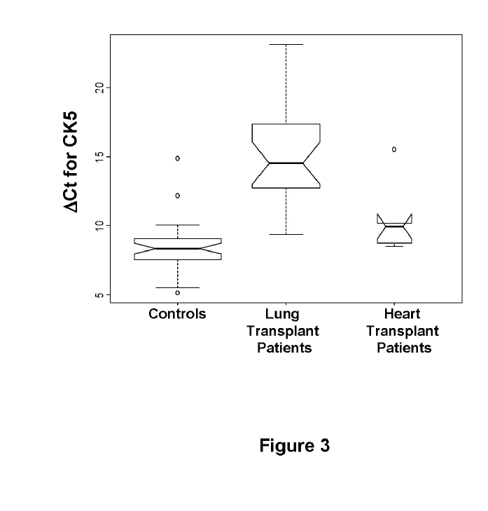

A 75-fold decrease was found in CK5 mRNA expression in the cohort of lung transplant patient samples tested post-transplant in comparison to healthy control subjects with 95% CI (33.36, 204.9) and p-value of 3.1×10⁻¹³ by the Wilcoxon rank sum test. This result is highly significant even after correcting for the multiple testing per patient, which would conservatively suggest a 0.005 significance level (for approximately 10 tests). FIG. 3 shows a boxplot comparison of ΔCt values between cases and controls.

All patients in the cohort were alive at more than one year post transplant, and to date none have developed bronchiolitis obliterans. No correlation in CK5 expression was found in patients with episodes of rejection or infection, although the study is not sufficiently powered to address this question. No significant difference in CK5 expression was found between patients with COPD compared to other airway diseases post-lung transplantation (p-value=0.106).

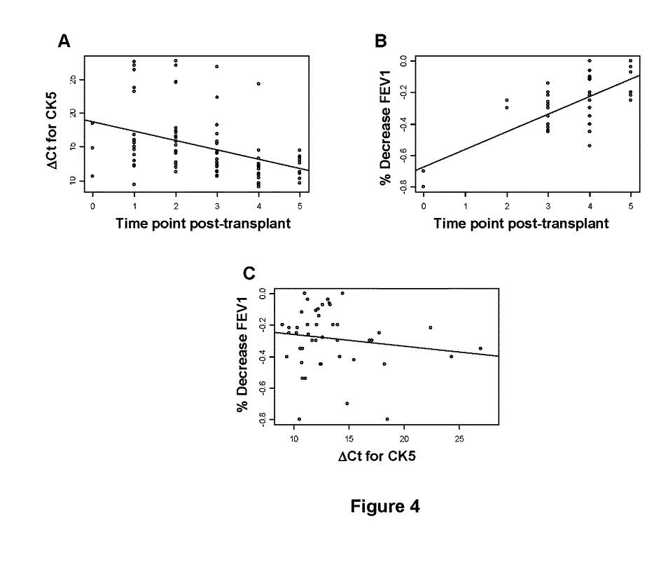

The inventors then examined patient outcomes by correlating their CK5 levels with time post-transplantation. CK5 mRNA expression was found to increase from the immediate post transplant period (within the first week when the donor graft has significant ischemic and reperfusion injury), to the latest available time point (typically 3-6 months post transplant) with a 9.2-fold increase in CK5 expression from the first CK5 level measured post-transplant to the last CK5 level post-transplant with 95% CT (2.42, 40.6) and a p-value of 1.4×10⁻⁵ for the correlation of CK5 mRNA expression and time post-transplant (FIG. 4A). As expected, a highly significant correlation was found between improvement in FEV1 and time post-transplantation (p-value=4.7×10⁻⁵) (FIG. 4B).

A two sample t-test comparing the CK5 levels (ΔCt) corresponding to the lower PFT readings and higher PFT readings (in patients without evidence of infection/rejection which could confound results) showed a significant difference in CK5 levels at the traditional 0.05 level (p-value=0.03). Since this comparison of ΔCt means is on the log scale for fold change it could imply a striking relationship between ΔCt and PFT in our data. The average difference: 15.98607−12.11172=3.87 corresponds to a fold change of 14.6 times less CK5 in the group with worse PFT's with corresponding 95% CI: (1.25, 171). This means that while on average we see a fold change of 14.6 less CK5, our results can not rule out fold changes as large as 171 times between the sicker and healthier PFT groups. FIG. 4C demonstrates the ΔCK5 mRNA expression values correlated with percentage decrease in FEV1.

In order to determine whether the reduction in circulating CK5 expression was specific to lung transplant patients, the inventors examined CK5 expression in the circulation of heart transplant patients. A significant difference was found between the CK5 expression levels in heart transplant patients and lung transplant patients, with the heart transplant patients having levels of CK5 expression in the circulation that were significantly greater than the lung transplant patients (p-value=0.004). There was an approximately 20.6 fold decrease in CK5 mRNA expression in lung transplant patients as compared to heart transplant patients (95% CI: 4.24, 191) (FIG. 3). However, there was a trend for heart transplant patients to express less CK5 in circulation than normal human subjects (p-value=0.05).

### Discussion

Building on the inventors' previous studies on CK5 expressing circulating epithelial progenitor cells in the circulation, the instant findings demonstrate the presence of CK5-positive cells in circulation in normal human subjects (Gomperts et al. (2006) J Immunol 176: 1916-1927). Furthermore, they correlate the levels of CK5 in circulation with lung status post transplant. The significant reduction in CK5 expressing cells in the circulation of lung transplant patients indicates that there may be a role for circulating epithelial progenitor cells in normal airway repair. This is further emphasized by the correlation that was found between an increase in circulating epithelial progenitor cells and an improvement in lung function after transplantation. Together, these results are consistent with the view that circulating epithelial progenitor cells is critical for normal airway repair and that a lack of circulating epithelial progenitor cells is associated with airway disease.

One limitation of the patient studies is the inability to determine the origin of the circulating epithelial progenitor cells. The results of this study suggest that the origin of CK5 expressing cells in circulation may be from the bone marrow, the thymus, or may be derived from the lung tissue itself, or may be derived from all of these compartments. If the origin of the circulating epithelial progenitor cells is the bone marrow, then it is possible that the immunosuppression the lung transplant patients are experiencing could be responsible for the decrease in the expression of CK5. The inventors therefore analyzed the expression of CK5 in the circulation of heart transplant patients who were also under immunosuppression but had no major airway epithelial injury. A significant difference was found in the amount of circulating epithelial progenitor cells between the heart transplant patients and lung transplant patients, which suggests that circulating epithelial progenitor cells are specific for airway repair. It was also noted that there is a trend in heart transplant patients towards having less circulating epithelial progenitor cells than normal human subjects. The heart transplant group were on lower doses of immunosuppression than the lung transplant patients, which suggests that immunosuppression may be playing a role in the reduction of circulating epithelial progenitor cells found in transplant patients.

The inventors did not find any significant difference in CK5 mRNA expression in normal human subjects with increasing age, as this might be expected as normal repair and regeneration decreases with age. However, there are likely many variables in addition to age that contribute to overall repair of the airway. CK5 expressing cells are also found as basal cells in other complicated epithelia, most notably skin and prostate, which could have led to the prediction that gender differences might be seen, although this was not the case in the control samples. However, as benign prostatic hypertrophy is associated with advancing age, it will be important in the future to examine CK5 levels in older normal human subjects to determine if there is a gender difference in this group.

Previous studies on the engraftment of bone marrow-derived epithelial cells in the distal airway after lung injury have shown conflicting results (Kotton et al. (2001) Development 128: 5181-5188; Kotton et al (2005) Am J Respir Cell Mol Biol 33: 328-334; Krause et al. (2001) Cell 105: 369-377; Mattsson et al (2004) Transplantation 78: 154-157; Suratt et al. (2003) Am J Respir Crit Care Med 168: 318-322; Kleeberger et al. (2003) Am J Pathol 162: 1487-1494). The results of this study demonstrate that circulating epithelial cells are present in the circulation of all normal human subjects examined. Flow cytometry analysis of CK5 expressing cells in circulation might be required to confirm this. The experiments in this study were performed retrospectively on frozen RNA samples and therefore flow cytometry for CK5 could not be performed. This study was not designed to assess the magnitude of engraftment of the circulating epithelial cells in the airway. Nonetheless, the statistically significant difference between lung transplant patients and normal human subjects with regard to their CK5 mRNA levels indicate that circulating epithelial cells are important in normal airway repair. In addition, the correlation of the increase in CK5 levels with the improvement in pulmonary function testing post-transplantation indicates that CK5 mRNA levels in circulation can be used as a biomarker of airway repair. It appears that circulating epithelial progenitor cells play an important role in airway repair, although the precise mechanism still needs to be established.

In summary, CK5 expressing circulating epithelial progenitor cells are present in circulation in normal human subjects and can be quantified with the real time PCR assay established by the present inventors. Furthermore, the circulating epithelial progenitor cells are significantly reduced immediately after lung transplantation, and then increase with time as lung function improves. These results show that circulating epithelial progenitor cells play an important role in airway repair after lung transplantation, and that circulating epithelial progenitor cells, as indicated by circulating CK5 mRNA level, may be used as a biomarker of airway repair.

### Example 2

### CK5 mRNA Level in Lung Cancer Patients

Lung cancer is the most deadly cancer worldwide. Current therapeutic strategies of chemotherapy, radiation, and trials with targeted therapies have only demonstrated extension in survival by a few months. Clearly, a novel approach is required to develop new therapies for this devastating disease. Cancer stem cells have been identified as the initial cell in the formation of carcinomas. They are capable of forming all the cell types of a tumor by dividing asymmetrically to form daughter cells as well as more differentiated cells resulting in the heterogeneity that is seen in tumors. Chemotherapy, radiation, and even targeted therapies are all designed to eliminate proliferating cells. However, cancer stem cells “hide out” in the quiescent phase of growth. This provides an explanation as to why our cancer therapies may produce an initial response but are often unsuccessful in curing patients. Lung cancer develops through a series of genetic and epigenetic changes that alter the epithelium from squamous metaplasia, then to dysplasia, carcinoma in situ and finally to invasive lung cancer. These changes are more likely to occur when cells proliferate rapidly. Cell proliferation is essential as a response to airway injury for epithelial repair, but excessive cell proliferation in response to injury can result in squamous metaplasia and ultimately transformation to cancer.

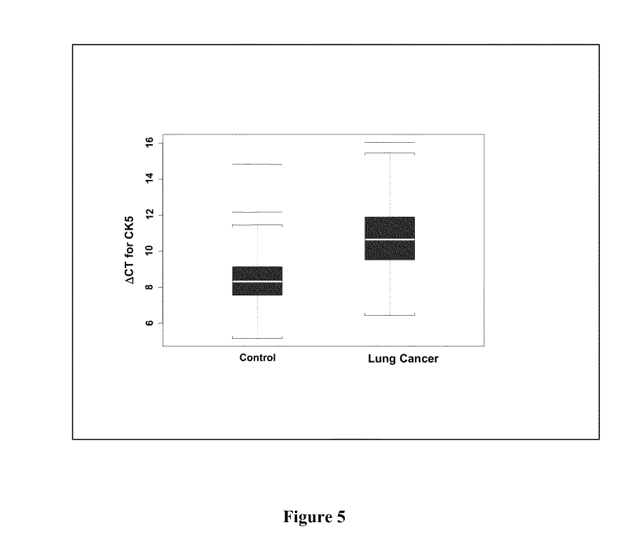

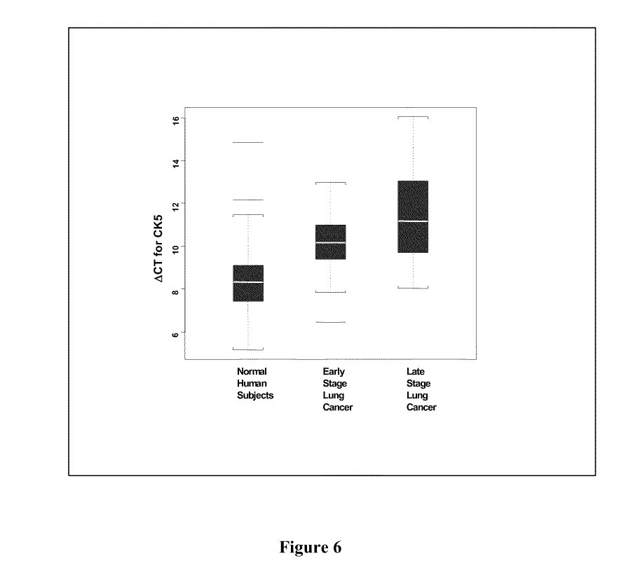

Normal repair of the airway epithelium occurs from progenitor/stem cells that reside in the submucosal glands/ducts and form the basal cells of the pulmonary epithelium. The basal cells express a progenitor epithelial cell marker cytokeratin 5 (CK5) and have been shown to have colony forming potential and the ability to form all the differentiated airway epithelial cell types. In addition to resident stem/progenitor cells, a population of circulating epithelial stem/progenitor CK5+ cells (CEPC) was identified in the bone marrow and circulation of mice that are also CXCR4+. Blocking these circulating epithelial progenitor cells with a neutralizing antibody to CXCL12 reduced trafficking of the circulating stem/progenitor epithelial cells into the injured airway and resulted in a phenotype of reserve cell hyperplasia and squamous metaplasia (FIG. 5). The hyperplasia and squamous metaplasia was solely derived from the resident stem/progenitor epithelial cells (FIG. 6). See, e.g., Gomperts et al. J Immunol. Feb. 1 2006; 176(3):1916-1927. This indicates that, in situations of airway injury, failure to recruit adequate numbers of circulating epithelial progenitor cells results in a default of the regenerating, proliferating epithelial cells to repair with the phenotype of squamous metaplasia.

The identification of CK5 expressing circulating epithelial stem/progenitor cells (CEPC) has revolutionized the thinking on how normal airway repair occurs in the lung and how lung cancer might arise. CEPC represent a novel reservoir of cells that can be mobilized at times of airway injury. Studies by the present inventors show that patients with lung cancer have lower levels of CEPC than normal human subjects (FIG. 5). In addition, it was discovered that patients with late stage (stage III/IV) lung cancer have lower CEPC levels than early stage (stage I/II) lung cancer patients (FIG. 6).

In one study by the inventors, the levels of CK5 mRNA were determined in 70 lung cancer patients (all stages of lung cancer), 38 normal human subject controls (same as in the lung transplant study), and 10 patients suffering from cancers other than lung cancer. As shown in Table 3, while there is no significant difference in the CK5 mRNA level between healthy subjects and non-lung cancer patients, the level of CK5 mRNA in lung cancer patients is significantly lower over non-lung cancer patients (p=0.01).

The most obvious immediate use for CEPC is therefore in lung cancer patients as a potential biomarker of recurrence and/or progression of disease. However, CEPC themselves could be of potential benefit because studies indicate that CEPC are critical for normal airway repair and that increasing their numbers could possibly prevent the initial steps in the development of lung cancer. CEPC therefore represent a unique population of cells that could have tremendous therapeutic benefits. Furthering understanding of the biology of resident stem/progenitor epithelial cells and CEPC in lung cancer is therefore critical for ultimately harnessing their regenerative potential. The goal of these studies is to advance the field towards clinical trials of stem cell based therapy for the prevention of lung cancer.

In addition, furthering understanding of the mechanisms underlying the transformation of the airway epithelium to squamous metaplasia has the potential to lead to the identification of new therapeutic targets and result in novel strategies to protect the repairing airway after injury such that the early precancerous stages of lung cancer can be prevented and therefore the lung cancer itself.

All patents, patent applications, and other publications cited in this application are incorporated by reference in the entirety for all purposes.

TABLE 1
Optimization of the Assay for Primers and Probes.

| [Primers (ea)] | [Probe] | Mean | CK5 | Mean |
|---|---|---|---|---|
| (nM) | (nM) | CK5 Ct | Ct SE | GAPDH Ct |
| 50 | 250 | 37.414 | 1.073 | 21.894 |
| 100 | 250 | 31.479 | 0.282 | 21.851 |
| 200 | 250 | 27.62 | 0.151 | 21.846 |
| 400 | 250 | 26.294 | 0.098 | 21.754 |
| 600 | 250 | 26.224 | 0.175 | 21.62 |
| 900 | 250 | 25.955 | 0.139 | 21.623 |
| 600 | 50 | 30.085 | 0.139 | 21.651 |
| 600 | 100 | 29.278 | 0.078 | 21.752 |
| 600 | 150 | 28.815 | 0.083 | 21.825 |
| 600 | 200 | 28.59 | 0.71 | 21.9 |
| 600 | 250 | 28.405 | 0.023 | 21.97 |

The Quantitative Real Time PCR assay was first performed with varying concentrations of primer and then probe to determine the optimal concentration of primers and probe for the assay. The concentration at which, for both primers and probe, the ΔCt and ΔRn did not change with increasing concentrations was selected. Thus primer concentrations of 600 nM and probe concentration of 250 nM were chosen.

TABLE 2
Lung transplant patient demographics.

| Patient # | Age (yr) | Gender | Diagnosis | Single/Double Lung |
|---|---|---|---|---|
| 1 | 58 | M | COPD | Double |
| 2 | 58 | M | COPD | Double |
| 3 | 64 | F | COPD | Double |
| 4 | 62 | M | PCH | Double |
| 5 | 61 | M | COPD | Double |
| 6 | 47 | F | sarcoid | Double |
| 7 | 60 | F | COPD | Double |
| 8 | 57 | M | IPF | Double |
| 9 | 31 | M | scleroderma | Double |
| 10 | 45 | F | PHTN | Double |
| 11 | 59 | M | IPF | Double |
| 12 | 63 | F | COPD | Single |
| 13 | 55 | F | ILD | Double |
| 14 | 60 | M | PHTN | Double |
| 15 | 53 | M | ILD | Double |
| 16 | 63 | F | IPF | Single |
| 17 | 55 | F | COPD | Double |
| 18 | 66 | F | COPD | Single |
| 19 | 56 | F | IPF | Double |
| 20 | 33 | M | scleroderma | Double |
| 21 | 79 | M | IPF | Single |
| 22 | 70 | M | IPF | Single |
| 23 | 62 | M | IPF | Double |

COPD = chronic obstructive pulmonary disease;
IPF = idiopathic pulmonary fibrosis;
PHTN = pulmonary hypertension;
ILD = interstitial lung disease;
PCH = Pulmonary capillary hemangiomatosis.

TABLE 3
CK5 mRNA Levels in Lung Cancer Patients

| | CK5 Exp (mean +/− SD) (Delta CT) |
|---|---|
| Normal | 8.5 +/− 1.7 |
| Lung Cancer | 10.3 +/− 1.8 |
| Early Lung Cancer | 9.8 +/− 1.4 |
| Late Lung Cancer | 10.8 +/− 2.2 |
| T1 + T2 | 10.0 +/− 1.6 |
| T3 + T4 | 11.1 +/− 2.7 |
| Other (not NSCLC | 8.7 +/− 2.1 |
| or Healthy Control) | |
| Lung Cancer | 10.3 +/− 1.8 |
| T1, N0, M0 (n = 17) | 9.6 +/− 1.3 |
| T2, N0, M0 (n = 14) | 9.9 +/− 1.5 |

Delta Ct values were analyzed, therefore lower values mean higher CK5 mRNA expression levels in circulation. The letters T, N, and M are used in accordance with the TNM staging system of lung cancer as described in Rami-Porta et al., Ann Thorac Cardiovasc Surg 15(1):4-9, 2009.

What is claimed is:

1. A method for detecting a lung disorder in a patient, the method comprising the steps of:
(i) quantitatively determining the amount of cytokeratin 5 (CK5) mRNA in the patient's blood; and
(ii) comparing the amount of CK5 mRNA from step (i) to a standard control representing the amount of CK5 mRNA in the blood of an average healthy person without any lung disorder, wherein an increase or decrease in the amount of CK5 mRNA from the standard control indicates the presence of a lung disorder.

2. The method of claim 1, wherein step (i) is performed by reverse transcriptase polymerase chain reaction (RT-PCR).

3. The method of claim 1, wherein a decrease in step (ii) indicates an injury of the lung.

4. The method of claim 1, wherein the decrease in the amount of CK5 mRNA from the standard control is more than 50%.

5. The method of claim 1, wherein the decrease in the amount of CK5 mRNA from the standard control is more than 20%.

6. The method of claim 1, wherein the decrease in the amount of CK5 mRNA from the standard control is more than 99%.

7. The method of claim 1, wherein a decrease in step (ii) indicates lung cancer.

8. The method of claim 1, wherein a decrease in step (ii) indicates a late stage lung cancer.

9. The method of claim 1, wherein the patient has received a lung transplant.

10. A kit for diagnosing or monitoring a lung disorder in a patient, the kit comprising:
(i) PCR primers for quantitatively determining the amount of CK5 mRNA in the patient's blood; and
(ii) a standard control representing the amount of CK5 mRNA an average healthy person without any lung disorder.

11. The kit of claim 10, further comprising user's instructions.

12. The kit of claim 10, wherein the lung disorder is an injury of the lung, a lung transplant, or a lung cancer.
